(19) United States
(12) Patent Application Publication
(10) Pub. No.: US 2008/0057019 A1
(43) Pub. Date: Mar. 6, 2008

(54) COMPOSITIONS AND METHODS FOR IMPARTING ODOR RESISTANCE AND ARTICLES THEREOF

(76) Inventor: Robert B. COLLIER, Cohutta, GA (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 11/850,062

(22) Filed: Sep. 5, 2007

Related U.S. Application Data
(60) Provisional application No. 60/824,699, filed on Sep. 6, 2006.

(51) Int. Cl. A61L 2/16

(52) U.S. Cl. 424/76.1; 252/182.35

(57) ABSTRACT

Described herein are compositions and methods for imparting odor resistance to an article. Also described herein are articles treated with the compositions and methods described herein.

### CROSS REFERENCE TO RELATED APPLICATIONS

This application claims priority upon U.S. provisional application Ser. No. 60/824,699, filed Sep. 6, 2006. This application is hereby incorporated by reference in its entirety for all of its teachings.

### BACKGROUND

Articles that are capable of efficiently removing chemical and other malodorous substances such as formaldehyde, acetaldehyde, ammonia, and acetic acid in the air have a number of applications. For example, carpet that is capable of removing odors produced from ammonia, trimethylamine, hydrogen sulfide, methyl mercaptan, acetic acid, and cigarette smoke is quite desirable.

Although the application of deodorants to articles such as carpet and textiles is known, these compositions possess various disadvantages. For example, the deodorant can be removed from the article by friction or washing and, thus, lose its deodorizing function quickly. This results in reduced efficiency of the deodorant composition. Furthermore, prior art deodorant compositions produce rigid fibers upon application to the fiber, which is also undesirable.

Thus, it would be desirable to produce a deodorant composition that upon application to an article is resistant to washing and wear and capable of maintaining its deodorizing function for a long time. It is also desirable that the deodorant composition be compatible with various additives and components. Finally, it is desirable that the resultant article be soft and not rigid after the article has been treated with the composition. The compositions described herein possess these advantages.

### SUMMARY

Described herein are compositions and methods for imparting odor resistance to an article. Also described herein are articles treated with the compositions and methods described herein. Additional advantages of the compositions, methods, and articles described herein will be set forth in part in the description that follows, and in part will be apparent from the description. The advantages of the compositions, methods, and articles described herein will be realized and attained by means of the elements and combination particularly pointed out in the appended claims. It is to be understood that both the foregoing general description and the following detailed description are exemplary and explanatory only and are not restrictive of the compositions, methods, and articles described herein, as claimed.

### DETAILED DESCRIPTION

The compositions, methods, and articles described herein can be understood more readily by reference to the following detailed description. It is also to be understood that the terminology used herein is for the purpose of describing particular aspects only and is not intended to be limiting.

It must be noted that, as used in the specification and the appended claims, the singular forms “a,” “an” and “the” include plural referents unless the context clearly dictates otherwise. Thus, for example, reference to “a zeolite” includes mixtures of zeolites.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc. of these materials are disclosed that while specific reference of each various individual and collective combination and permutation of these compounds may not be explicitly disclosed, each is specifically contemplated and described herein. For example, if a hydrophobic material is disclosed and discussed and a number of different zeolites are discussed, each and every combination and permutation of the hydrophobic material and the zeolite that are possible are specifically contemplated unless specifically indicated to the contrary. For example, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Throughout this application, where publications are referenced, the disclosures of these publications in their entireties are hereby incorporated by reference into this application in order to more fully describe the state of the art to which this invention pertains.

In one aspect, any of the compositions described herein can be used to impart odor resistance. In one aspect, the composition includes (a) a hydrophobic material and (b) a zeolite.

The term “hydrophobic material” is defined herein as any material that increases the hydrophobicity of a substrate (e.g., a fiber) after the material has been applied to the substrate. The hydrophobic material can exist in a variety of forms including, but not limited to, resins, waxes, polymers, and the like. In one aspect, the hydrophobic material comprises the hydrophobic material comprises a wax-modified polymer, a phenolic resin, a fluorochemical, a binder, or any combination thereof.

The term “wax-modified polymer” is defined herein as a compound composed of a wax component and a polymer component, wherein the wax component and polymer component are covalently attached to one another. The term “wax-modified polymer” is also defined herein as a compound composed of a blend of a wax component and a polymer component. In one aspect, the wax component contains a group that can react with an amino group or a hydroxyl group. In one aspect, the wax component can be paraffin. In one aspect, any of the waxes disclosed in U.S. Pat. No. 4,566,980, which is incorporated by reference in its entirety, can be used herein as the wax component. In one aspect, the wax includes one or more of a natural wax or a synthetic wax. In one aspect, the natural wax includes animal wax (e.g., beeswax, lanolin, shellax wax, Chinese insect wax) or a mineral wax (e.g., fossil or earth waxes such as ozocerite, ceresin, or montan, or petroleum waxes such as paraffin or microcrystalline wax). In another aspect, the synthetic wax can be a polyalkylene such as an ethylenic polymer and polyol ether-esters such as Carbowax and sorbitol, a chlorinated naphthalene such as Halowax, or a hydrocarbon produced from a Fischer-Tropsch reaction.

In one aspect, the polymer component of the wax-modified polymer contains an amino group or a hydroxyl group. In one aspect, the polymer can be a melamine resin, a phenolic acid resin, a urea resin or a combination thereof. Any of the melamine resins and derivatives thereof disclosed in U.S. Pat. Nos. 5,952,447; 6,040,044, and 6,534,150 B1, which are incorporated by reference in their entireties, can be used herein. In one aspect, two or more different polymers can be used to prepare the wax-modified polymer. In one aspect, the polymer component can be an acrylate, a methacrylate, a formaldehyde condensate, or a urea condensate. In one aspect, the wax-modified polymer is AQUASTAB (a low molecular weight, oxidized polyolefin wax) manufactured by Eastman Chemical Company. In another aspect, the wax-modified polymer is CEROL-EX manufactured by Clariant, which is the reaction product between paraffin and melamine resin.

In one aspect, the hydrophobic material comprises a phenolic resin produced by the condensation between formaldehyde and a hydroxyl aryl sulfone. For example, the phenolic resin can be produced by condensing formaldehyde with bis-(hydroxyphenyl)sulfone, phenyl sulfonic acid, 2,2 bis-(hydroxyphenyl)propane, bis-(hydroxyphenyl)ether-dihydroxy diphenyl sulfone, or benzene sulfonic acid. Phenolic resins are generally referred to in the art as novolac resins.

In one aspect, the hydrophobic material is a fluorocompound. In one aspect, the fluorocompound can include, but is not limited to, fluorochemical urethanes, ureas, esters, ethers, alcohols, epoxides, allophanates, amides, amines (and salts thereof), acids (and salts thereof), carbodiimides, guanidines, oxazolidinones, isocyanurates, and biurets. Blends of these compounds are also considered useful. In another aspect, the fluorocompound can be a fluoropolymer. Examples of fluoropolymers useful herein include, but are not limited to, fluorinated acrylate and substituted acrylate homopolymers or copolymers containing fluorinated acrylate monomers interpolymerized with monomers free of non-vinylic fluorine such as methyl methacrylate, butyl acrylate, acrylate and methacrylate esters of oxyalkylene and polyoxyalkylene polyol oligomers (e.g., oxyethylene glycol dimethacrylate, polyoxyethylene glycol dimethacrylate, methoxy acrylate, and polyoxyethylene acrylate), glycidyl methacrylate, ethylene, butadiene, styrene, isoprene, chloroprene, vinyl acetate, vinyl chloride, vinylidene chloride, vinylidene fluoride, acrylonitrile, vinyl chloroacetate, vinylpyridine, vinyl alkyl ethers, vinyl alkyl ketones, acrylic acid, methacrylic acid, 2-hydroxyethylacrylate, N-methylolacrylamide, 2-(N,N,N-trimethylammonium)-ethyl methacrylate, and 2-acrylamido-2-methylpropanesulfonic acid (AMPS). In another aspect, the fluoropolymer can be a urethane backbone fluoropolymer, wherein the fluoropolymer is cationic, anionic, or neutral. An example of an anionic urethane backbone fluoropolymer useful herein is ZONYL N-119 manufactured by Du Pont or ARROWTEX F10-X manufactured by Arrow Engineering.

In another aspect, the hydrophobic material comprises one or more binders. A “binder” as used herein is any material that facilitates the bonding of one or more components present in the composition (e.g., zeolite) to the article. The binders can be used in emulsions or hot melt adhesives. In one aspect, the binder can be a polymeric resin. The binders disclosed in U.S. Pat. Nos. 4,775,588; 5,147,722; and 5,539,015, which are incorporated by reference in their entireties, can be used herein. In one aspect, the binder can be a polyolefin (e.g., polyethylene, polypropylene, polybutene-1, and poly-4-methylpentene-1); a polyvinyl (e.g., polyvinyl chloride, polyvinyl fluoride, and polyvinylidene chloride); a polyurethane; a polyacrylate (e.g., polyacrylate or polymethacrylate); a polyvinyl ester (e.g., polyvinyl acetate, polyvinyl propionate, and polyvinyl pyrrolidone); a polyvinyl ether; a polyvinyl sulfate; a polyvinyl phosphate; a polyvinyl amine; a polyoxidiazole; a polytriazol; a polycarbodiimide; a copolymer or block interpolymer (e.g., ethylene-vinyl acetate copolymer); a polysulfone; a polycarbonate; a polyether (e.g., polyethylene oxide, polymethylene oxide, and polypropylene oxide); a polyarylene oxide; a polyester (e.g., a polyarylate such as polyethylene terephthalate); an isocyanate; or a polyimide. In one aspect, the binder can be Colloid 137 (methacrylate polymer) manufactured by Kemira or Flexipel SR-30 or Flexipserse SB-400 manufactured by Innovative Chemical Technologies.

In one aspect, the binder comprises a metallocene polyethylene or polypropylene. Metallocene polyethylene or polypropylene is prepared by the polymerization of ethylene or propylene in the presence of a metallocene catalyst. Techniques for producing metallocene polyolefins useful herein are disclosed in U.S. Pat. Nos. 6,407,189; 6,331,590; and 6,080,902. In one aspect, the metallocene polyethylene and polypropylenes sold under the tradename Licocene manufactured by Clariant can be used herein. Examples of such materials include Licocene PE 4201, PE 3401, PP 6102, PP 4202, PP 2602, PP 1302, PP 1502, PP 1602, PP 7502, and PP 6402 (PE is polyethylene and PP is polypropylene). In certain aspects, the metallocene polyethylene or polypropylene can be functionalized with one or more different groups. For example, the metallocene polyethylene or polypropylene can be functionalized with an anhydride group or silane group. The polymers are generally produced by polymerizing unsaturated monomers containing the anhydride or silane group with ethylene or propylene. Examples of modified metallocene polyethylene or polypropylene include Licocene PE MA 4221, PE MA 4351, PE SI 3262, PP MA 6252, PP MA 1332, PP SI 3361, PP MA 7452, and PP MA 6452 (PE MA is polyethylene modified with maleic anhydride; PP MA is polypropylene modified with maleic anhydride; PE SI is polyethylene modified with silane groups; and PP SI is polypropylene modified with silane groups).

In one aspect, the hydrophobic material is a UV-curable coating or material. In another aspect, the hydrophobic material is a blocked polyisocyanate, which is stable at room temperature but dissociates to regenerate isocyanate at elevated temperatures. In a further aspect, the hydrophobic material is a polyurethane coating. For example, the polyurethane can be a waterborne coating derived from polyurethane dispersions or blocked polyisocyanates. Examples of polyurethanes useful herein include, but are not limited to, Bayhydrol®, Impranil®, Baybond®, and Bayhydur® manufactured by Bayer. In another aspect, the hydrophobic material is low density polyethylene, low density polypropylene, high density polyethylene, high density polypropylene, styrene butadiene, nylon, isoprene, latex rubber, nitrile, nitrile rubber, silicone, polycarbonate, polyisoprene, polystyrene, rubber, polyvinyl acetate, a polyethylene/ polypropylene acrylic, a polytetrafluoroethylene acrylic, a polyethersulfone acrylic, a polycarbonate, or any combination thereof.

The amount of hydrophobic material present in the composition will vary depending upon the article to be treated as well as the particular hydrophobic material and zeolite that are used. In one aspect, the hydrophobic material can be from 1% to 99% by weight, 1% to 90% by weight, 1% to 80% by weight, 1% to 70% by weight, 1% to 60% by weight, 1% to 50% by weight, 1% to 40% by weight, 1% to 30% by weight, 1% to 20% by weight, 5% to 15% by weight, or 10% by weight. In another aspect, the hydrophobic material can be used in dry form or in the form of an emulsion or dispersion.

The zeolite used in the compositions described herein can include any zeolite known in the art. The zeolite can be natural or synthetic. Not wishing to be bound by theory, it is believed that the zeolite imparts the odor resistance when the composition is applied to the article. Depending upon the zeolite selected, the zeolite can mask, neutralize, absorb, or reduce odors that can be present on the article. Alternatively, the zeolite can remove odor-causing molecules by catalysis (e.g., convert the odor-causing molecule to a non-odorous molecule) or ion exchange. The mechanisms described above are not intended to be limiting or exhaustive.

In general, zeolites are aluminosilicate materials. Any of the zeolites disclosed in U.S. Pat. Nos. 4,304,675; 4,437,429; 4,793,833; and 6,284,232 B1, which are incorporated by reference in their entireties, can be used herein. Examples of zeolites useful herein include, but are not limited to, amicite, analcime, barrerite, bellbergite, bikitaite, boggsite, brewsterite, chabazite clinoptilolite, cowlesite, dachiardite, edingtonite, epistilbite, erionite, faujasite, ferrierite, garronite, gismondine, gmelinite, gobbinsite, gonnardite, goosecreekite, harmotome, herschelite, heulandite, laumontite, levyne, maricopaite, mazzite, merlinoite, mesolite, montesommaite, mordenite, natrolite, offretite, paranatrolite, paulingite, perlialite, phillipsite, pollucite, scolecite, sodium dachiardite, stellerite, stilbite, tetranatrolite, thomsonite, tschemichite, wairakite, wellsite, willhendersonite, yugawaralite, or any combination thereof.

In one aspect, zeolites useful herein include Max Pell (natural zeolite) manufactured by Mountain Minerals; Advera™ 201 (sodium aluminosilicate), Advera™ 401P (aluminosilicate), and Turbobrite™ (aluminosilicate) manufactured by PQ Corporation; Agriculite™ manufactured by Agricola Materials Corp.; ZEOCAT® FM-8 (sodium form of mordenite) and ZEOCAT® FM-8/25 (hydrogen form of mordenite) manufactured by Zeochem AG; LZM-8 powder (synthetic hydrogen form of aluminosilicate zeolite mordenite) manufactured by Customtec; REMASIL® and MULGRAIN™ manufactured by Remet® Corp.; HiSiv™ 1000 and 3000 (high silica zeolite) manufactured by UOP LLC; ZeoPro™ manufactured by ZeoponiX; and MagicMineral (laumontite) manufactured by Zeo-Tech Corp. Zeolites manufactured by Minerals Research; Tricat Zeolites GmbH; Enviro Corp.; Badger Mining Co.; GSA Resources, Inc.; Sinanen Zeomic, Ltd.; Zeo, Inc.; Victor Industries, Inc.; Cyclotrol Diversified Industries, Inc.; ASH Meadows Zeolite, LLC; Zeolyst International; Natural Zeolites Industry; and Steelhead Specialty Minerals can also be used herein.

In one aspect, the zeolite includes a mixture of SiO₂, Al₂O₃, and Na₂O. In one aspect, when the zeolite includes a mixture of SiO₂, Al₂O₃, and Na₂O, the amount of SiO₂ present is from 70% to 99% by weight, 80% to 99% by weight, 90% to 99%, 90% to 95% by weight, or 92% to 95% by weight of the zeolite; the amount of Al₂O₃ in the zeolite is from 1% to 20% by weight, 2% to 10% by weight, 3% to 7% by weight, or from 4% to 6% by weight of the zeolite; and the amount of Na₂O in the zeolite is from 0.5% to 20% by weight, 1% to 10% by weight, 1% to 8% by weight, 1% to 6% by weight, 1% to 4% by weight, or from 1% to 2% by weight of the zeolite. In another aspect, the zeolite has the formula Naₓ(AlO₂)_{y}(SiO₂)_{y}—XH₂O, where x is greater than 1, y is greater than or equal to x, and X is greater than 1. In one aspect, the zeolite can be mordenite. In another aspect, mordenite manufactured by Chemie Uetikon and P.Q. Corp. can be used herein.

The amount of zeolite present in the composition will vary depending upon the article to be treated as well as the particular zeolite that is used. In one aspect, the zeolite can be from 1% to 99% by weight, 1% to 90% by weight, 1% to 80% by weight, 1% to 70% by weight, 1% to 60% by weight, 1% to 50% by weight, 1% to 40% by weight, 1% to 30% by weight, 5% to 30% by weight, 10% to 25% by weight, or 20% by weight of the composition.

In one aspect, the zeolite has an average particle size less than 10 microns. In other aspects, the average particle size is less than 9 microns, less than 8 microns, less than 7 microns, less than 6 microns, less than 5 microns, less than 4 microns, less than 3 microns, less than 2 microns, or less than 1 micron. Techniques for producing zeolite with small particle sizes include grinding larger particles of zeolite to a desired particle size. For example, wet grinding zeolite can produce particles having diameters from 3 to 4 microns. Alternatively, dry grinding the particles can produce particle sizes approaching 1 micron. Examples of machines useful for producing small zeolite particles include, but are not limited to, a vertical bead mill, a horizontal bead mill, or a triple cowls blade. By modifying the particle size of the zeolite, the odor resistant properties can be increased. Smaller particle sizes increase the surface area of the zeolite dispersed in the composition, which increases odor resistance. Additionally, the use of smaller zeolite particles facilitates the production of the compositions described herein. In certain aspects, when the zeolite is mixed with the hydrophobic material, the mixture is cured to form a matrix, where the zeolite particles are entrapped within the matrix. The curing step generally involves the application of heat. In the case when smaller zeolite particles are used, the curing step can be performed at ambient conditions. Thus, the use of expensive heating equipment is not needed, which reduces overall production cost.

In one aspect, any of the zeolites described herein can be encapsulated. The term “encapsulated” is defined herein as zeolite particles contained within or surrounded by a thin layer of polymer (e.g., by the binder). Thus, encapsulation involves entrapping the zeolite particle within a polymeric coating. Alternatively, the term “encapsulated” can also include zeolite particles entrapped within a polymer matrix. Polymers suitable for use in making polymer-encapsulated zeolites are homopolymers or random and block copolymers produced by free-radical, ionic, or coordination polymerization of one or more polymerizable monomers. Generally, the polymers are natural or synthetic polymers made by addition or condensation polymerizations. Examples include polystyrenes, polyolefins, polyureas, polyacrylics, polyurethanes, polyesters, polyamides, fluorinated polymers, polysaccharides, polypeptides, polynucleotides, and the like, and mixtures thereof. The polymers can be generated by bulk, solution, suspension, or emulsion polymerization methods. The polymers can be hydrocarbons, or they can incorporate functional groups such as hydroxyl, amine, phosphine, phosphine oxide, arsine, sulfur, sulfur oxides, fluoroalkyl, alkoxy, silane, siloxy, carboxy, or the like. The polymer can be the same or different as the polymer that is used to make the fiber to be coated.

There are many suitable ways to encapsulate zeolites within a polymer. Some of these techniques have been used to encapsulate pharmaceuticals to mask taste, impart storage stability, or target drug delivery; others have been used to encapsulate solid pesticide particles. Suitable techniques include, for example, spray-drying, spray-chilling, spray-coating, phase separation and coascervation, injection treatment coating, fluid bed coating, dry-on-dry coating, melt extrusion, vapor deposition, in situ polymerization, including in situ interfacial polymerization, and the like. These and other microencapsulation techniques are described in the introductory chapter of Microcapsules and Nanoparticles in Medicine and Pharmacy, M. Donbrow, Ed., pp. 1-14, and references cited therein, and in G. Beestman, “Microencapsulation of Solid Particles,” Controlled-Release Delivery Systems for Pesticides (1999), H. Scher, Ed., pp. 31-54. U.S. Pat. Nos. 6,156,245 and 6,958,405 also discloses useful techniques.

The term encapsulated zeolite also covers the use of a zeolite as an encapsulation device for other materials. For example, catalysts known in the art to convert odor-causing molecules to non-odorous molecules can be imbedded or incorporated in a zeolite lattice or matrix. In one aspect, one or more metallophthalocyanines can be encapsulated into the pores of the zeolite.

Depending upon the application of the composition, the composition can contain various other additives and components. In one aspect, the composition can optionally include a surfactant. Examples of surfactants include, but are not limited to, dispersants, emulsifiers, detergents, and wetting agents. Any of the surfactants disclosed in U.S. Pat. Nos. 4,648,882 and 5,683,976, which are incorporated by reference in their entireties, can be used herein.

In one aspect, the surfactant is anionic, cationic, or neutral. In one aspect, the anionic surfactant can be a sulfate and sulfonate, although other types, such as soaps, long-chain N-acyl sarcosinates, salts of fatty acid cyanamides or salts of ether carboxylic acids, of the type obtainable from long-chain alkyl or alkylphenyl poly-ethylene glycol ethers and chloroacetic acid, can also be used. The anionic surfactant can be used in the form of the alkali metal or alkali earth metal salt.

In one aspect, surfactants of the sulfate type can be sulfuric acid monoesters of long-chain primary alcohols of natural and synthetic origin containing from 10 to 20 carbon atoms, i.e. of fatty alcohols such as, for example, coconut oil fatty alcohols, tallow fatty alcohols, oleyl alcohol, or of C₁₀-C₂₀ oxoalcohols and those of secondary alcohols having chain lengths in the same range. Sulfated fatty acid alkanolamides and sulfated fatty acid monoglycerides are also suitable.

In another aspect, surfactants of the sulfonate type can be a salt of sulfosuccinic acid monoesters and diesters containing from 6 to 22 carbon atoms in the alcohol portions, alkylbenzene sulfonates containing C₉-C₁₅ alkyl groups and lower alkyl esters of α-sulfofatty acids, for example the α-sulfonated methyl or ethylesters of hydrogenated coconut oil fatty acids, hydrogenated palm kernel oil fatty acids or hydrogenated tallow fatty acids. Other suitable surfactants of the sulfonate type are the alkane sulfonates obtainable from C₁₂-C₁₈ alkanes by sulfochlorination or sulfoxidation and subsequent hydrolysis or neutralization or by addition of bisulfites onto C₁₂-C₁₈ olefins and also the olefin sulfonates i.e. mixtures of alkene and hydroxyalkane sulfonates and disulfonates, obtained for example from long-chain monoolefins containing a terminal or internal double bond by sulfonation with gaseous sulfur trioxide and subsequent alkaline or acidic hydrolysis of the sulfonation products.

In one aspect, the surfactant can be a disodium alpha olefin sulfonate, which contains a mixture of C₁₂ to C₁₆ sulfonates. In one aspect, CALSOFT™. AOS-40 manufactured by Pilot Corp. can be used herein as the surfactant.

The amount of surfactant present in the composition will vary depending upon the article to be treated as well as the particular surfactant that is used. In one aspect, the surfactant can be from 1% to 20% by weight, 1% to 10% by weight, 1% to 8% by weight, 1% to 6% by weight, or 5% by weight of the composition.

In another aspect, any of the compositions described herein can optionally include one or more metal oxides other than zeolite. In one aspect, the metal oxide can be a transition metal oxide. In another aspect, the metal oxide is an oxide of silicon, aluminum, titanium, zirconium, zinc, or a combination thereof. The amount of metal oxide present in the composition will vary depending upon the article to be treated as well as the particular metal oxide, hydrophobic material, and zeolite that are used. In one aspect, the metal oxide can be from 1% to 20% by weight, 1% to 10% by weight, or 5% to 10% by weight of the composition. Not wishing to be bound by theory, it is believed that the metal oxide behaves as a catalyst and regenerates the zeolite so that the zeolite can continue to impart odor resistance to the article.

In another aspect, any of the compositions described herein can optionally include (1) an anionically modified phenol formaldehyde polymer comprising a phenol moiety and a formaldehyde moiety, (2) a naphthalene condensate, (3) a lignin sulfonate, (4) a phenol sulfonate derivative, or a mixture thereof. Any of the anionically modified phenol formaldehyde polymers, naphthalene condensates, lignin sulfonates, and phenol sulfonate derivatives disclosed in U.S. Pat. No. 6,387,448 B1, which is incorporated by reference in its entirety, can be used herein.

The anionically modified phenol formaldehyde polymers appropriate for use in the compositions described herein include, but are not limited to, condensation products of aldehydes with phenyl bearing molecules and anionically modifying agents. The phenol formaldehyde polymer can be anionically modified by methods including, but not limited to, sulfonation, phosphonation and acylation. When sulfonation is desired, it can be accomplished by the use of sulfonic acid. In one aspect, the polymer contains phenylsulfonic acid residues. In other aspects, the polymer can be a condensation product of naphtholsulfonic acid and an aldehyde, an anionically modified hydroxyaromatic formaldehyde condensate, the condensation product of anionically modified dihydroxydiphenylsulfone or the condensation product of naphtholsulfonic acid or the derivatives of any of these polymers.

Examples of other suitable anionically modified phenol formaldehyde polymers or compounded materials based on phenol formaldehyde polymers include, but are not limited to, DU PONT SR-500 (Du Pont), FX 369, 668, 661 (3M), INTRATEX N (Crompton and Knowles), ERIONYL PA (Ciba-Geigy), NYLOFIXAN P and PM (formerly Sandoz, now Claraint), MESITOL NBS (formerly Mobay Chemical Corp., now Dystar, Inc.), ARROWSHIELD™ GSR AND ARROWSHIELD™ 2713 (Arrow Engineering), etc. In an alternative aspect, lignin sulfonates can be used in place of the anionically modified phenol formaldehdye polymer. In yet another aspect, naphthalene condensates can be used in place of the anionically modified phenol formaldehyde polymer. In yet another aspect, phenol sulphonate derivatives can be used in place of the anionically modified phenol formaldehyde polymer.

Compounds suitable for use as the anionically modified phenol formaldehyde polymer are disclosed in U.S. Pat. Nos. 4,592,940; 4,839,212; 4,822,373; 4,940,757; and 4,937,123, which are herein incorporated by this reference in their entirety and for the teachings of suitable anionically modified phenol formaldehyde polymers.

In another aspect, any of the compositions described herein can optionally include an aluminum polymer. The term “aluminum polymer” is defined as any polymeric material that contains at least one aluminum atom. The aluminum atom in the aluminum polymer can be covalently or ionically attached to the polymeric material. In one aspect, the polymeric material can contain at least one group that can interact with the aluminum atom either by a Lewis acid/base interaction or a Bronsted acidibase interaction. Examples of polymeric materials that can be used to produce the aluminum polymer include, but are not limited to, polyesters, polyols, polyamines, polyamides, polyurethanes, polycarbonates, polyacrylates, polymethacrylates, or a melamine-based resin. In one aspect, the polymeric material used to produce the aluminum polymer does not have any fluoro atoms or groups containing fluoro atoms covalently attached to the polymeric material. The molecular weight of the polymeric material can vary depending upon the polymer selected and its application.

The aluminum polymers can be prepared using techniques known in the art. For example, polyacrylic acid can be treated with a base to deprotonate at least one carboxylic acid group followed by the addition of an aluminum compound such as, for example, an aluminum salt, to produce aluminum polyacrylate. In one aspect, the aluminum polymer can be aluminum polyacrylate, aluminum polymethacrylate, or a combination thereof. For example, aluminum polyacrylate and aluminum polymethacrylate provided by Aldrich Chemical Company can be used herein. In one aspect, aluminum polyacrylate and aluminum polymethacrylate can be prepared from the polymerization of aluminum acrylate and aluminum methacrylate, respectively, using techniques known in the art.

The aluminum polymer can be used in various forms including, but not limited to, a solid (e.g., a powder) or a dispersion (e.g., in water or organic solvent). The amount of aluminum polymer present in the composition will vary depending upon the article to be treated as well as the particular aluminum polymer that is used.

In addition to the components discussed above, the compositions described herein can include other ingredients including, but not limited to, anionic leveling agents, cross-linking agents, optical brighteners, chelating agents, and inorganic/organic salts, foaming agents, ultra-violet absorption, enhanced lightfastness, flame retardants, odor elimination products, fillers and carriers, antisoiling or resoiling inhibitors, preservatives, thickeners, etc.

In one aspect, the compositions described herein do not contain a polyester. In another aspect, any of the compositions described herein optionally do not contain an amine compound or a hydrazine compound, wherein the amine compound has a particle diameter less than or equal to 20 μm. For example, any of the amine compounds and hydrazine compounds disclosed in U.S. Pat. No. 6,335,075 are not used in the compositions described herein. In another aspect, the composition is substantially in the absence of an amine compound or a hydrazine compound, wherein the amine compound has a particle diameter less than or equal to 20 μm. The phrase “substantially in the absence of” is defined herein as a composition having less than 0.5% by weight, less than 0.25% by weight, less than 0.1% by weight, less than 0.05% by weight, less than 0.025% by weight, or less than 0.001% by weight of the amine compound or hydrazine compound.

In one aspect, the composition consists essentially of the hydrophobic material and the zeolite. In this aspect, it is contemplated that the composition contains small amounts of other components such, where these components do not affect one way or the other the odor-resistant properties of the composition.

In one aspect, the composition includes paraffin-melamine resin as the hydrophobic material and mordenite as the zeolite. In another aspect, this composition optionally contains disodium alpha olefin sulfonate as the surfactant and/or zinc oxide as the metal oxide.

In another aspect, the composition includes (a) a hydrophobic material and (b) activated carbon. In one aspect, the activated carbon can be derived from coconut shells.

Any of the compositions described herein can be produced by admixing the hydrophobic material and the zeolite or activated carbon, and one or more optional ingredients discussed above in any order. The term “admixing” is defined as the mixing of two or more components together so that there is no chemical reaction or physical interaction. The term “admixing” also includes the chemical reaction or physical interaction between any of the components described herein upon mixing to produce the composition.

The components used to produce the compositions described herein can be admixed using techniques described in the art. For example, mixers such as paddle mixers, drum mixers, auger mixers and the like can be used. In one aspect, finely divided solid constituents are initially introduced into the mixer in which they are then sprayed while mixing with the liquid constituents. In another aspect, either the solid components and/or the liquid components are premixed prior to their introduction into the mixer. In one aspect, after thorough blending of the finely divided solid constituents with the liquid constituents, a smooth flowable powder or liquid is produced.

In one aspect, any of the compositions described herein can be applied to an article using techniques known in the art. The method for contacting the article with the composition will vary depending upon the article and the form of the composition. In one aspect, the compositions described herein can be in the form of an aqueous medium or a dispersion, such as a foam. Alternatively, the compositions described herein can be dissolved or dispersed in an organic solvent such as, for example, a glycol or polyether, or an aqueous organic solvent. In this aspect, the composition can be applied to the article by spray application. In another aspect, other methods such as, for example, Beck application, Continuous Liquid and Foam application, Flood, Flex Nip, Pad, and Superba (saturated steam continuous heat setting) applications can be used to contact the article with the composition.

In another aspect, when the contacting step involves topical coating, the coating step can be performed by spray, foam, kiss or liquid injection methods and various methods thereof followed by drying in a hot air or radiant heat oven at 160 to 320° F. for a time sufficient to dry the article. In one aspect, a spray application can be applied in a liquid medium (water and chemical treatment) with a wet pickup of 5% to about 200% followed by drying. In another aspect, a foam application can be applied in a liquid medium (water and chemical treatment) with a wet pickup of 5% to about 200%. In this aspect, the foam can be applied by a direct puddle application with a press roll, an injection manifold and/or a sub-surface extraction device. Subsequent drying in a hot air or radiant heat oven at 160 to 320° F. for a time sufficient to dry the article should follow.

The prevailing plant conditions will also affect the amount of composition to be applied to the article to achieve the desired odor resistance. The composition of the article will also influence the amount of composition to be applied.

Application conditions such as pH, temperature, steam and drying time can vary. In one aspect, the pH range for the compositions described herein is from about 1.0 to about 11.0. Still further, the pH of the compositions of the present invention can be from 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5 or 11.0 where any value can be used as an upper or a lower endpoint, as appropriate. As would be recognized by one of ordinary skill in the art, the amount of pH adjustment needed prior to use of the compositions will depend on the amount of each component in the composition. Further, pH adjustment of the composition prior to use can be by methods known to one of ordinary skill in the art, such as the addition of acid or base, as appropriate.

The temperature at which the article is contacted by the compositions described herein range from ambient to temperatures up to 100° C. at atmospheric pressure and above 100° C. under pressure conditions (closed atmosphere). Still further, the temperature of application can be from 25, 35, 45, 55, 65, 75, 85 or 100° C., where any value can form an upper or a lower end point, as appropriate. In another aspect, the composition can be cured at ambient temperature once applied to the substrate.

Where production procedures warrant, steam can aid in the efficacy of the compositions herein when applied by, but not limited to Beck, Continuous liquid, Flood, Flex Nip, Superba, and Pad applications. The steam time can vary from about 15 seconds to about 10 minutes, or from about 2 minutes to about 8 minutes. Still further, the application time can be from about 15 seconds or 1, 2, 3, 4, 5, 6, 7, 8, 9 or 10 minutes, where any value can form an upper or a lower end point, as appropriate. In certain applications, but not limited to Spray Application and Foam Application, drying with forced heat can aid in the fixing of the composition to the article. In one aspect, the coated article can be dried with forced air. In another aspect, the coated article can be dried with microwave heat. The drying time is generally dependent upon varying conditions predicated by moisture content, range speed, type construction, the weight of the substrate, etc. The drying time can vary from 30 seconds to 15 minutes. Still further, the drying time can be from 15 seconds or 1, 3, 5, 7, 9, 10, 12, or 15 minutes, where any value can be used as an upper or lower endpoint, as appropriate.

In one aspect, the weight ratio of the composition can vary between 0.5% to 600% of wet pick up where such amount is based on the weight of the article and the composition that is used. The weight ratio will vary dependent on the manner of application. In other aspects, the owf (“on weight fiber”) amount of the composition that can be applied to the article is from 0.05, 0.1, 0.2, 0.5, 1, 2, 3, 4, 5, 6, 7, 8, 9, 10, 30, 50, 70, 100, 120, 150, 200, 250, 300, 350, 400, 450, 500, 550 or 600% as measured by weight of the article, where any value can be used as an upper or lower endpoint, as appropriate. In one aspect, the owf amount of the composition that is applied to the article is from 0.5% to 5.0%.

In one aspect, once the article has been contacted with the composition, the article can be further treated to remove any composition that is not bound to the article.

Also contemplated are articles treated with any of the compositions described herein. In one aspect, the article can be composed of any material that can receive and that will adhere to the composition where odor-resistance is desirable. Examples of articles include, but are not limited to, bedding (e.g., blankets, sheets, pillowcases, futon or comforter covers, comforter wadding), clothes (e.g., suits, uniforms, shirts, blouses, trousers, skirts, sweaters, socks, panty hoses, shoe linings, shoe sole inserts), curtains, carpet, diapers, incontinent pads, surgical sponges and dressings, surgical pads, or catamenial devices such as sanitary napkins, shields, liners, or tampons.

In one aspect, the article is composed of natural and/or synthetic fibers. In one aspect, the synthetic fiber includes, but is not limited to, polyamide fibers, polyester fibers, polypropylene fibers, synthetic fibers containing free amino groups, and derivatives thereof such as nylon covered with polypropylene. Fibers containing free amino groups can be obtained by a variety of methods, including, but not limited to, the condensation reaction of hexamethylenediamine with adipic acid, hexamethylenediamine with sebacic acid, ξ-aminodecanoic acid, caprolactam and dodecylcaprolactam. Fibers formed from polyaryl amides, including type 6 and type 6,6 nylons, can be treated by the compositions and methods described herein. Examples of natural fibers include, but are not limited to, cotton, wool, and flax. Semisynthetic fibers such as rayon can also be contacted with any of the compositions described herein. In one aspect, the fibers are Dupont's Antron®, Sorona® yarn manufactured by Dupont, and Corterra® (polytrimethylene terephthalate) manufactured by Shell Chemicals.

The fibers treated with the compositions and methods described herein can be twisted, woven, tufted and sewn into various forms of textile materials including, but not limited to, rugs, carpets, and yams. The fibers can be treated and then formed into the various forms of textile materials, or the formed textile can be treated.

In one aspect, the article can contain one or more fluorocompounds prior to treatment with any of the compositions described herein. In this aspect, these articles are referred to herein as fluorinated articles, wherein the article has at least one fluoro group. In one aspect, when the article is a fiber, the fluorocompound can be extruded with the material used to make the fiber so that the resultant fiber contains the fluorocompound incorporated throughout the fiber. Any of the fluorocompounds described above can be used in this aspect. The number or amount of fluoro groups present in the fluorinated article will vary depending upon the article and the fluorocompound selected. In one aspect, the amount of fluoro groups present in the fluorinated article can be from 20 ppm to 5,000 ppm, 50 ppm to 5,000 ppm, 100 ppm to 5,000 ppm, 150 ppm to 5,000 ppm, 200 ppm to 5,000 ppm, 200 ppm to 4,000 ppm, 200 ppm to 3,000 ppm, 200 ppm to 2,000 ppm, or 200 ppm to 1,000 ppm.

In one aspect, the compositions described herein can impart odor resistance to an article. The term “odor resistance” is defined herein as the ability of the compositions described herein to neutralize, absorb, or reduce odors that can be present on or near the article. The term “odor resistance” is also defined herein as the ability of the compositions described herein to chemically convert malodorous molecules to molecules that have no odor or reduced odor. For example, the compositions described herein can chemically degrade an organic molecule to smaller, non-odorous molecules.

In another aspect, any of the zeolites described herein can be extruded with the polymer used to make a fiber. In this aspect, the zeolite, which can also be encapsulated, is incorporated throughout the fiber. The method generally involves (a) admixing a polymer used to make the fiber and zeolite, and (b) extruding the mixture produced in step (a) to produce the fiber. The extrusion of polymers to produce fibers is well-known in the art. These techniques can be applied in the methods described herein. In other aspects, any of the odor-resistant compositions described herein can be admixed with the polymer used to make the fiber prior to extrusion.

Throughout this application, various publications are referenced. The disclosures of these publications in their entireties are hereby incorporated by reference into this application in order to more fully describe the compounds, compositions and methods described herein.

Various modifications and variations can be made to the materials, methods, and articles described herein. Other aspects of the materials, methods, and articles described herein will be apparent from consideration of the specification and practice of the materials, methods, and articles disclosed herein. It is intended that the specification and examples be considered as exemplary.

What is claimed:

1. A composition comprising (a) a hydrophobic material and (b) a zeolite, wherein the hydrophobic material comprises a wax-modified polymer, a binder, or a combination thereof, and the composition does not contain a polyester.

2. The composition of claim 1, wherein the wax-modified polymer comprises a blend of a wax and a polymer.

3. The composition of claim 2, wherein the wax comprises paraffin.

4. The composition of claim 2, wherein the polymer comprises one or more of a phenolic resin or a urea resin.

5. The composition of claim 2, wherein the polymer comprises a melamine resin or a derivative thereof.

6. The composition of claim 1, wherein the hydrophobic material comprises a paraffin-melamine resin.

7. The composition of claim 1, wherein the binder comprises a polyolefin, a polyvinyl, a polyurethane, a polyacrylate, a polyvinyl ester, a polyvinyl ether, a polyvinyl sulfate, a polyvinyl phosphate, a polyvinyl amine, a polyoxidiazole, a polytriazol, a polycarbodiimide, a copolymer or block interpolymer, a polysulfone, a polycarbonate, a polyether, a polyarylene oxide, a polyimide, or a combination thereof.

8. The composition of claim 1, wherein the binder comprises a metallocene polyethylene or polypropylene.

9. The composition of claim 8, wherein the metallocene polyethylene or polypropylene is functionalized with an anhydride group or silane group.

10. The composition of claim 1, wherein the hydrophobic material comprises a wax-modified polymer and a binder.

11. The composition of claim 1, wherein the hydrophobic material further comprises a phenolic resin, a fluorochemical, or a combination thereof.

12. The composition of claim 1, wherein the zeolite has an average particle size less than 10 microns.

13. The composition of claim 1, wherein the zeolite comprises mordenite.

14. The composition of claim 1, wherein the zeolite is encapsulated.

15. The composition of claim 1, wherein the composition further comprises one or more of a metal oxide or the salt thereof, wherein the metal oxide is not a zeolite.

16. The composition of claim 1, wherein the composition further comprises a solvent.

17. The composition of claim 16, wherein the solvent comprises an organic solvent, water, or a combination thereof.

18. The composition of claim 1, wherein the composition further comprises an anionically modified phenol formaldehyde polymer comprising a phenol moiety and a formaldehyde moiety, a naphthalene condensate, a lignin sulfonate, a metal oxide, an aluminum polymer, or a combination thereof.

19. The composition of claim 1, wherein the composition is substantially in the absence of a hydrazine compound or an amine compound, wherein the amine compound has a particle diameter less than or equal to 20 μm.

20. The composition of claim 1, wherein the composition does not contain a hydrazine compound or an amine compound, wherein the amine compound has a particle diameter less than or equal to 20 μm.

21. A method for imparting odor-resistance to an article, comprising contacting the article with the composition of claim 1.

22. An article comprising the composition of claim 1.

23. A fiber made by the process comprising (a) admixing a polymer used to make the fiber and zeolite, and (b) extruding the mixture produced in step (a) to produce the fiber.

24. The fiber of claim 23, wherein step (a) comprises admixing the polymer with the composition of claim 1.
